(19) United States
(12) Patent Application Publication
(10) Pub. No.: US 2004/0228910 A1
(43) Pub. Date: Nov. 18, 2004

(54) Transdermal, oral and intravenous formulations of 2, 3-dimethoxy-5-methyl-6-decaprenyl-1, 4-benzoquinone

(75) Inventors: Franz Enzmann, Bad Homburg (DE); Burkhard Lachmann, Rotterdam (NL)

Correspondence Address: JACOBSON HOLMAN PLLC, 400 SEVENTH STREET N.W., SUITE 600, WASHINGTON, DC, 20004, US

(73) Assignee: MSE Pharmazeutika GmbH

(21) Appl. No.: 10/783,029

(22) Filed: Feb. 23, 2004

Related U.S. Application Data
(63) Continuation of application No. 09/355,931, filed on Oct. 12, 1999, now abandoned.
Application No. 09/355,931 is a 371 of international application No. PCT/EP98/00744, filed on Feb. 11, 1998.

(30) Foreign Application Priority Data
Feb. 11, 1997 (DE) 197 05 231.2

(51) Int. Cl. A61K 9/127; A61K 31/12

(52) U.S. Cl. 424/450; 514/690

(57) ABSTRACT

Transdermal, oral and intravenous formulations of 2,3-dimethoxy-5-methyl-6-decaprenyl-1,4-benzoquinone (coenzyme Q10), containing an effective amount of pulmonary surfactant, also in combination with liposomes, in addition to usual excipients.

2,3-Dimethoxy-5-methyl-6-decaprenyl-1,4-benzoquinone is also known by the designation of coenzyme Q10. This substance plays a role in the respiratory chain and, in addition, is an antioxidant which is capable of scavenging free radicals, which are transmitted by vitamins, in particular. In addition, Q10 determines the elasticity and dynamics of cell membranes. Therefore, it is recommended as a monopreparation and in combination with other active substances for oral administration. For skin care, it is additionally offered in the form of a liposome cream which allows the active ingredient to penetrate through the horny layer barriers and then to accumulate in the various strata of the skin. The liposome cream used to date has been prepared on the basis of lecithins, forming a lipid bilayer around an aqueous interior space. Q10 deposits inside the membrane.

It has now been found that transdermal, oral and intravenous formulations of 2,3-dimethoxy-5-methyl-6-decaprenyl-1,4-benzoquinone can be improved and made more effective if they contain an effective amount of pulmonary surfactant in addition to the usual excipients. Pulmonary surfactant is a complex of phospholipids, neutral lipids and surfactant proteins which together form a monolayered barrier between the air and the liquid surface of the lung. Pulmonary surfactant is produced in the alveolar type II cells from which it is released into the alveolar space.

Since pulmonary surfactant is released from the alveolar type II cells into the air cavity of the lungs, it was not considered that pulmonary surfactant might penetrate into tissue layers. Therefore, to date, pulmonary surfactant has only been employed for instillation in diseases or deficiencies of the lung, and for the transport of antibiotics and corticosteroids into the lung.

Other applications have not been considered to date. It has now been found unexpectedly that pulmonary surfactant is capable of penetrating into the outer skin and the mucosa of the gastrointestinal region, the oral and vaginal regions, i.e., either alone or in combination with liposomes.

It is of minor importance whether highly purified or less highly purified pulmonary surfactant preparations from a wide variety of species or pulmonary surfactant obtained by recombination are employed (pig, cow, sheep, etc.). Less highly purified preparations have the advantage of a low-cost production.

Since any strained tissue has a more or less pronounced Q10 deficiency, it has been tried to transport Q10 into the inadequately supplied regions with the aid of pulmonary surfactant. A combination of liposomes and pulmonary surfactant has actually proven advantageous.

Thus, the formulation according to the invention containing 2,3-dimethoxy-5-methyl-6-decaprenyl-1,4-benzoquinone and an effective amount of pulmonary surfactant can be very successfully employed for the oral treatment of diseases of the cardiovascular system, the lung, the muscles, the stomach and bowels (ulcer and gastritis), diabetes, the skin, the nerves, tinnitus, in degenerative metabolic imbalance, incontinence, periodontosis, mitochondrial diseases, immune deficiency and rheumatism. In addition it has been established that this combination according to the invention can also be successfully employed for the topical treatment of psoriasis, neurodermitis, burns, radiolesions, eczemas, wounds, ulcus cruris, cancer of the skin and skin ageing.

1-5. (canceled)

6. A method of rendering skin or mucosa permeable to 2,3-dimethoxy-5-methyl-6-decaprenyl-1,4-benzoquinone (Q10) comprising administration to the skin or mucosa of a patient a therapeutic formulation containing Q10 and an effective amount of protein-containing pulmonary surfactant in combination with excipients.

7. The method of claim 6, characterized in that the therapeutic formulation further comprises liposomes.

8. The method of claim 6, characterized in that the protein-containing pulmonary surfactant employed is a raw extract from an animal species.

9. The method of claim 6, characterized in that administration is to oral mucosa.

10. The method of claim 6, characterized in that administration is to pulmonary mucosa.

11. The method of claim 6, characterized in that administration is to gastrointestinal mucosa.

12. The method of claim 6, characterized in that administration is to vaginal mucosa.

13. The method of claim 6, characterized in that administration is to the skin.

14. A method of rendering skin or mucosa permeable to 2,3-dimethoxy-5-methyl-6-decaprenyl-1,4-benzoquinone (Q10) comprising administration to the skin or mucosa of a patient a therapeutic formulation containing Q10 and an effective amount of a complex of phospholipids, neutrolipids, and surfactant proteins.

15. The method of claim 14, characterized in that the therapeutic formulation further comprises liposomes.

16. The method of claim 14, characterized in that the protein-containing pulmonary surfactant employed is a raw extract from an animal species.

17. The method of claim 14, characterized in that administration is to oral mucosa.

18. The method of claim 14, characterized in that administration is to pulmonary mucosa.

19. The method of claim 14, characterized in that administration is to gastrointestinal mucosa.

20. The method of claim 14, characterized in that administration is to vaginal mucosa.

21. The method of claim 14, characterized in that administration is to the skin.
